(19) United States
(12) Patent Application Publication
(10) Pub. No.: US 2012/0270293 A1
(43) Pub. Date: Oct. 25, 2012

(54) STANDARDIZATION OF TISSUE SPECIMEN PRESERVATION BY ULTRASOUND AND TEMPERATURE CONTROL

(76) Inventor: Wei-Sing Chu, Silver Spring, MD (US)

(21) Appl. No.: 13/514,046

(22) PCT Filed: Dec. 1, 2010
(86) PCT No.: PCT/US10/58521

§ 371 Date: Jun. 5, 2012

Related U.S. Application Data
(60) Provisional application No. 61/283,558, filed on Dec. 7, 2009.

(51) Int. Cl. C12N 13/00; C12M 1/42; C12N 5/07

(52) U.S. Cl. 435/173.5; 435/374; 435/307.1

(57) ABSTRACT

A tissue sample preservation process and device for improving and standardizing tissue preservation procedure, including placing tissue specimens in a cold fixative, performing fixative penetration at a refrigerated temperature, and accelerating fixative penetration by ultrasound. Also disclosed is the application of ultrasound and temperature control in the dehydration, clearing, and impregnation steps.

### BACKGROUND OF INVENTION

1. An Overview of the Field of Tissue Preservation

Immediately after a tissue specimen is removed from patient body, the ischemic cascade begins. The ischemic cascade leads to changes of many susceptible cellular biomolecules such as mRNA degradation and protein dephosphorylation in the tissue specimen. Therefore the longer the ischemia, the more pre-analytical changes will happen in the patient tissue specimens: Such changes often hamper correct and sensitive molecular diagnosis and prognosis assays [Liotta 2000, Emmert-Buck 2000, Compton 2007, Hewitt 2008, Espina 2008]. Flash-freezing by which tissue specimens are preserved in a deep frozen state to prevent biomolecule changes, is a standard protocol to preserve tissue specimens for molecular analysis. However, in addition to the high cost and sophistication of the equipment, flash-freezing destroys the morphology of the tissue specimens. Good tissue morphologies are paramount requirements for diagnostic tissue samples.

In the current practice of surgical histology, tissue specimens are placed, bulk or grossed, into a fixative for fixation. Fixed tissues are then processed through dehydration, clearing, paraffin impregnation, and embedded in a paraffin block. Formalin is the most commonly used fixative by the clinical pathology community [Hewiit 2008, Fox 1985, 1987, Boon 1988]. Modern histology is based on the tissue morphologies produced by the formalin fixed and paraffin embedded (FFPE) tissues. Formalin fixation is carried out at room temperature overnight or longer in most clinical pathology laboratories. Susceptible biomolecules may degrade before formalin fully diffuses into the specimen. Reactions between formaldehyde and proteins happen quickly at room temperature, causing extensive cross-linking formation in the tissue periphery and little cross-linking formation in the tissue center. Prolonged fixation time is sometimes required for the center area to catch up with peripheral area in the extent of cross-linking [Medawar 1941; Boon 1988; Helander 1994, 1999; Ruijter 1997].

The formalin fixed tissue samples have the following 2 major problems: (1) biomolecules are heavily modified by extensive cross-linking; and, (2) due to varied fixation times, cross-linking levels vary in different samples. Therefore conventional FFPE tissue samples are generally not standardized. A lack of standardization and heavy modification of biomolecules are the major obstacles preventing FFPE tissue samples from being used in quantitative molecular analysis.

A number of none cross-linking fixatives are developed in attempts to accommodate quantitative molecular analysis [Wenk 2006; Wester K 2003; Boon 2008; Espina 2009]. Tissue morphologies produced by non cross-linking fixatives are different from those produced by formalin—a major factor hampering wide acceptance of non cross-linking fixatives in the clinical pathology community. An ideal solution for tissue preservation problems is to develop a method that can generate both the gold standard FFPE morphology and high quality biomolecules, comparable to those from flash-frozen tissues, in a simple and cost effective way.

2. Description of the Related Art

Conventional methods prepare tissues for histology by incubation in separate solutions of phosphate-buffered 10% formaldehyde for fixation, a series of increasing concentrations of ethanol for dehydration, and xylene for clearing tissue of dehydration agent, prior to impregnation with paraffin. Because of the time required for this process, usually 8 hours or longer, it is customary to complete these separate steps—fixation, dehydration, clearing, and impregnation—overnight in automated mechanical instruments designed for those tasks (see, for example, U.S. Pat. Nos. 3,892,197, 4,141,312, and 5,049,510). A typical automated tissue processor (TISSUE-TEK) requires more than eight hours and is programmed to process batches of tissue samples.

In water solution, formaldehyde molecules [HCOH] is hydrated to form methylene glycol [CH₂(OH)₂] which exists at equilibrium with unhydrated formaldehyde molecules, as shown below:

HCOH+H₂O [symbol] CH₂(OH)₂

Low temperature tilts the equilibrium to methylene glycol molecules which penetrate tissues much faster than unhydrated formaldehyde molecules. However, it is the formaldehyde molecules that form the cross-linking bridge. There is a conundrum in the formalin fixation in relation with temperatures: 1) Low temperature favors biomolecule preservation in tissue specimens, slows down cross-linking, facilitates even diffusion of formalin molecules throughout tissue specimens; 2) High temperature favors HCOH formation which in turn facilitated cross-linking, while cross-linking of proteins in tissue periphery slows down formalin penetration into the tissue center; 3) At a low temperature speed of molecule diffusion in general is low due to reduction in molecule motion and leads to significant reduction in penetration speed for formalin molecules.

At room temperature, the sequence of the events for formalin fixation of a routine tissue specimen (1-4 mm in thickness) is as follows: (1) Rapid diffusion of methylene glycol to reach the interior requires approximately 1-4 h; (2) The succeeding steps (dehydration of methylene glycol and cross-linking reaction) together require approximately 24 h. Therefore, diffusion of methylene glycol in this case is not hindered by a dense network of cross-linked proteins. However, at high temperatures, all three steps are accelerated. Because dehydration and cross-linking also are completed first in the parts of the heated tissue where methylene glycol is present, i.e., the periphery of the tissue block, further diffusion into the center is hindered by the dense protein network thus created.

To accelerate tissue processing, U.S. Pat. Nos. 4,656,047, 4,839,194, and 5,244,787 use microwave energy; U.S. Pat. Nos. 3,961,097, 5,089,288, and 6,291,180 use ultrasonic energy; and U.S. Pat. No. 5,023,187 uses infrared energy.

Three factors of ultrasound that can influence tissue fixation are as following: 1) microcavitations and highly efficient convection produced by ultrasound in the fixative and the tissue samples increase tissue permeability to fixative molecules; 2) ultrasound causes gradual temperature increase leading to gradual increase in cross-linking speed; 3) ultrasound may generate free radicals in the fixative which also favor cross-linking formation.

As the personalized medicine is looming, there is an urgent need for high quality and standardized tissue specimens. This need, when translated into the FFPE tissue sample preparation, requires that tissues being fixed quickly and evenly, and processing steps standardized. Therefore, it is desirable to establish procedures and possibly instruments to achieve all these.

Lack of standardization for protocols as well as lack of on-site quality control in tissue fixation by formalin are among the major drawbacks for the FFPE tissue samples. The long time involved in the fixation, especially that of large specimens is a major limitation in making a timely diagnosis. It is also considered a major factor affecting the incentives to change the pre-fixation tissue handling procedures in both the operating rooms and pathology laboratories. In most hospitals, the tissues are placed in formalin in the operating room; therefore, the pathologist cannot control fixation time. Accordingly, it is virtually impossible to standardize quantitative assays on paraffin sections, because the duration of formalin fixation influences the degree of cross-linking which in turn affects the availability of biomolecules for quantitative assays. In addition, solid specimens larger than 25 gram are not always completely fixed after being submerged in formalin for 24 hr. Cutting the surgical specimen into smaller pieces facilitates fixation, but is undesirable for final anatomical orientation and often delays pre-fixation time.

Changes of macromolecule composition, especially cell components involved in cell signaling pathways, as well as tissue autolysis, happen immediately after tissue is removed from patients. In protocols used by most pathological departments formalin fixation is done at room temperature or even higher to facilitate formalin penetration. During the prolonged penetration stage at ambient temperatures, autolytic degradation of susceptible biomolecules, such as mRNA, phosphorylated proteins, and protein antigens may occur. In addition to macromolecule changes, progressive formalin fixation at ambient temperature causes prolonged exposure to formalin at the periphery, leading to over fixation, while fixation is incomplete at the center of large specimens. This uneven fixation often leads to inconsistent results in immunohistochemistry assays and possibly many other molecular tests.

### SUMMARY OF THE INVENTION

The invention is about processes and devices that are used in the fixation and processing of tissue specimens facilitated by ultrasound and temperature control to achieve standardized tissue specimen preservation. First of all, the invention relates to a low temperature fixation procedure which comprises: (a) submerging tissue specimens in a fixative at a refrigerated cold temperature; (b) irradiating the tissue specimens in the fixative with ultrasound with a superimposing cooling system to keep the temperature of the tissue specimen and fixative low; (c) optionally, turning off the superimposing cooling system and raise temperature of the tissue specimens and the fixative by continued ultrasound irradiation alone or in combination with another heating means; and, (d) optionally, stopping (quenching) the cross-linking reaction by chemical reagents or cooling.

The invention also relates to a tissue specimen preservation procedure, comprising: (a) a fixation step which comprises: i) tissue specimens are submerged in fixative immediately after tissue specimens are excised from patients and kept at cold temperature until the next step in the process; ii) tissue specimens in cold fixative are irradiated by ultrasound and the specimen/fixative temperature is kept low by a cooling system superimposed on the ultrasound irradiation for thorough and uniform penetration of fixative molecules; iii) optionally, the fixative temperature is then raised by ultrasound, microwave, or energy of other kinds, when the cooling system is turned off; (b) at least one dehydration step which comprises submerging fixed tissue specimen in a dehydration solution and irradiating the fixed tissue specimen with ultrasound; (c) at least one clearing step which comprises submerging the fixed and dehydrated tissue specimen in a clearing solution and irradiating the fixed and dehydrated tissue specimen with ultrasound; (d) an impregnating step which comprises submerging the fixed, dehydrated, and cleared tissue specimen in melted wax or paraffin and irradiating the fixed, dehydrated, and cleared tissue specimen with ultrasound.

The invention also relates to a tissue specimen preservation device comprising at least one vessel for holding tissue specimens and preservation reagents, an ultrasound generating system to produce ultrasound field in tissue preservation reagents, and a temperature control system that is coupled with the ultrasound generating system for maintaining a cold temperature.

### BRIEF DESCRIPTION OF THE DRAWINGS

FIG. 1. Tissue specimen preservation workflow for standardization of pre-analytical tissue specimen preservation by ultrasound and temperature control.

FIG. 2. Cross-linking effect of fixative solutions with different formaldehyde concentrations.

FIG. 3. Lysozyme, BSA, myoglobin, RNase A, trypsin, and egg white were each dissolved (or diluted) in 1× PBS, pH 7 at 20 mg/ml. Aliquots of 50 micro liter of each protein solution were mixed with equal volumes of NBF and incubated at designated temperatures for 10 minutes. After incubation, 6 micro liter of each incubation mixture was mixed with 2 micro liter of 4× SDS loading buffer and loaded in a well on SDS PAGE, separated by electrophoresis, and stained with Coomassie blue.

FIG. 4. One configuration of US chamber in which US transducer is included in a transducer housing removable from the solution chamber (reaction chamber). In this configuration, sound wave is emitted in one direction. Tissue specimens may be removably attached to the housing and can be moved together with the housing from one reaction chamber to the next reaction chamber.

FIG. 5. One configuration of US chamber in which US transducer is included in a transducer housing removable from the solution chamber (reaction chamber). In this configuration, sound wave is emitted in two directions. Tissue specimens may be removably attached to the housing and can be moved together with the housing from one reaction chamber to the next reaction chamber.

FIG. 6. One configuration of US chamber coupled with a cooling/heating system. In this configuration, fixative or processing solutions are cooled or heated in a cooling/heating device and circulated in and out of the US chamber.

FIG. 7. One configuration of a cylinder/piston device to generate vacuum and pressure in the US chamber. In this configuration, the bottom part of the US chamber which is affixed with a US transducer moves up and down to produce pressure and vacuum in the US chamber.

FIG. 8. One figuration of a cylinder/piston device to generate vacuum and pressure in the US chamber. In this configuration, the top cover of the US chamber moves up and down to produce pressure and vacuum in the chamber.

FIG. 9. Comparison of H&E staining of bovine kidney (1), liver (2), and pancreas (3) tissues fixed at (A) room temperature overnight and (B) 4° C. (upper panel); and the quantification of number of nuclei per field as well as nuclear size (lower panel).

FIG. 10. H&E staining of center and periphery of liver tissue fixed with and without US irradiation.

FIG. 11. IHC and Western blot studies on cow kidney tissue fixed for 30 min at 4 C with US irradiation (US-LT-FFPE) and for overnight at room temperature (FFPE). IHC assays against vimentin and cytokeratin were done without antigen retrieval. Western blot assays were done with whole protein extracts from same amounts of tissue samples fixed with respective method.

FIG. 12. The comparison of inhibition activity to RNase A at RT by Formalin with, RNA Later, 50% methanol, and in PBS with autoclave (120° C.) for 30 minutes.

FIG. 13. RNase A at designated amount was incubated with NBF at RT or 4° C. for 5 min and then mixed with 2 μg yeast tRNA and incubated at room temperature for 30 min for RNase A digestion and separated by 2% agarose gel. The tRNA molecules were between 50-100 nucleotides in length.

FIG. 14. SDS PAGE and Western blot analysis on lysates of T47D cells fixed in neutral buffered formalin at 4 C or RT for various lengths of time. Left: SDS PAGE stained by Coomassie Blue.

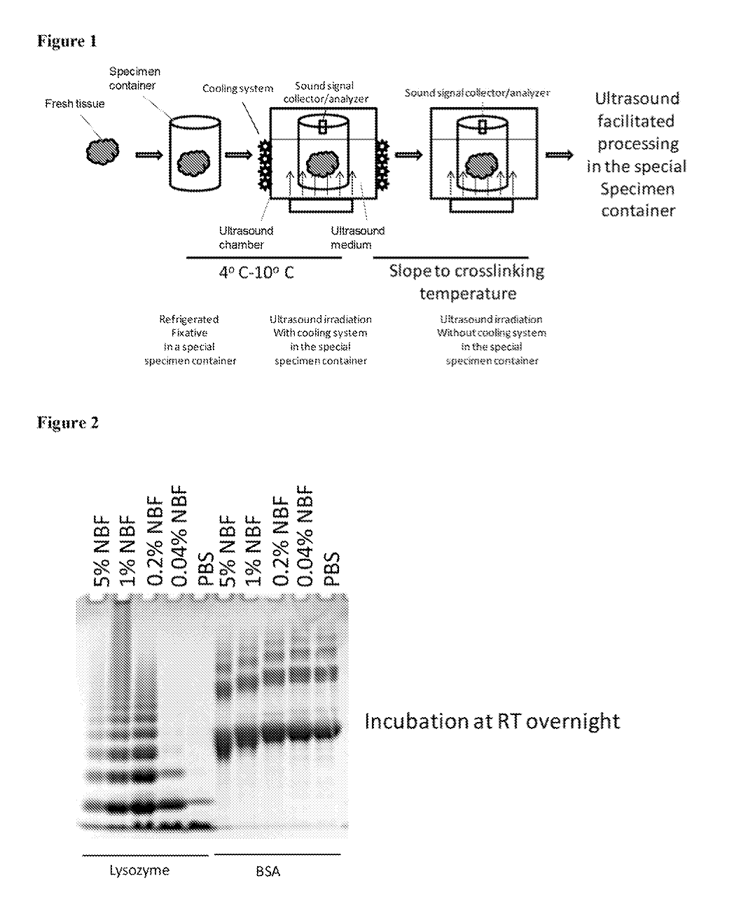

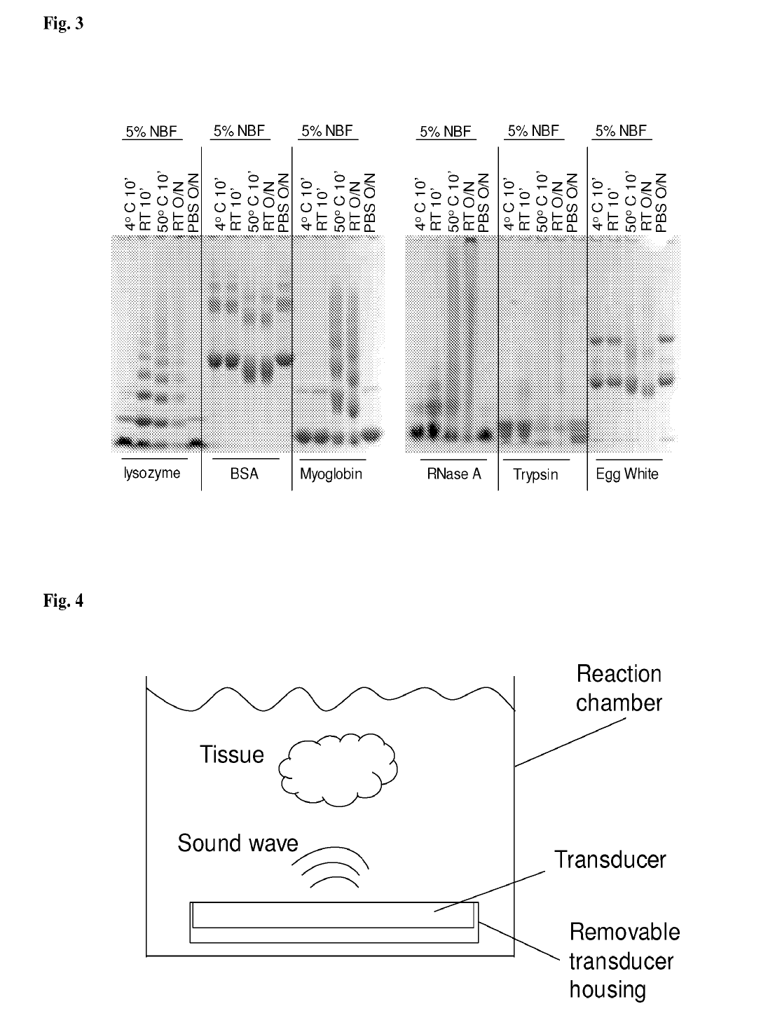

Middle: Western blot membrane probed with pooled antibodies against HER-2 and ER. Right: Plots for digital intensities of HER-2 and ER Western blot signals against fixation time.

### DETAILED DESCRIPTION OF THE INVENTION

Currently, almost all clinical tissue specimens are fixed in formalin at room temperature [Hewitt 2008, Boon 2008]. Fixing tissues in cold formalin is considered time consuming and used occasionally for research purposes. The obstacles for wide acceptance of the cold formalin fixation procedure in the clinical community include the following:

1. It is commonly believed that an adequate cross-linking level must be reached in the tissues to be fixed to ensure proper preservation of microscopic details.

2. It takes much longer for tissue specimens to reach the desired cross-linking level at cold temperature than at room temperature.

3. It is more convenient to fix tissue specimens at room temperature.

We have shown that tissue specimens fixed by formalin at 4 C overnight generated excellent FFPE morphology, although the tissue specimens still looked fresh, indicating that a marginal level of cross-linking was sufficient to produce the gold standard formalin morphology. The integrity and availability of biomarkers in the cold-formalin-fixed tissues were better than those in tissues fixed by the conventional room temperature method for immunohistochemistry (INC) and Western blot analysis. We further demonstrated that ultrasound (US) irradiation could significantly reduce time needed in fixation of tissue specimens in formalin at cold temperature.

A good tissue specimen preservation strategy should demonstrate the ability to instantly inhibit autolysis (i.e., inactivation of endogenous destructive enzymes), the ability to minimize biomolecule modifications, the ability to produce preserved tissue samples of best cellular and subcellular morphology, and the ability for as much as possible macromolecules to be recovered from preserved tissue samples.

Neutral buffered formalin (NBF), often referred to as 10% NBF, is a 10-fold dilution of saturate formaldehyde solution in phosphate buffered saline (PBS), and has the formaldehyde concentration of about 3.7-4%. NBF is a commonly used cross-linking fixative in routine tissue preservation. Solutions with formaldehyde concentration lower than 4%, e.g., 2%, 1% and even lower were tested for their cross-linking effect to proteins. The lower the formaldehyde concentration is the less effective is the fixative solution in forming cross-linking or modifications, thus cross-linking or biomolecule modification level is often low.

Disclosed here is a method to control and standardize the cross-linking/modification levels in tissue fixation by lowering the concentration of fixative agents. Compared with NBF (e.g., 3.7%-4% formaldehyde), penetration of formaldehyde solution of lower concentration into tissue specimen is slower. However, facilitated by ultrasound and/or other means to increase permeability of tissue specimens, fixative solutions with lower formaldehyde concentration can be used and less toxic formaldehyde waste will be generated. Since less cross-linking and other biomolecule modifications are formed, solutions with lower formaldehyde concentrations can further facilitate biomolecule recovery from fixed tissue samples as well as standardization of tissue fixation by producing a uniform cross-linking/modification level throughout the tissue specimen.

Also disclosed is a method to control the cross-linking/modification level, as well as slowing the tissue auto degradation, during tissue fixation by low temperature. At a cold temperature, biomolecule cross-linking/modification by formaldehyde is slow, so a uniform and moderate level of cross-linking/modification can be readily achieved throughout the tissue specimen. Since enzymatic activities are greatly reduced at cold temperatures, ischemic cascade and tissue degradation will be greatly reduced. A cold temperature (e.g., 0 degree C. to 25 degree C., preferably 4 C to 20 C, further preferably 4 C to 18 C) can be applied throughout fixation step. It can also be applied as a first penetration phase which is followed by warm cross-linking phase. Or it can even be applied at a sequence of cold phase-warm phase-cold phase, where the second cold phase is used to stop or reduce the cross-linking speed in the warm phase.

Another method to control and standardize the cross-linking/modification levels is by addition of quenching (stopping) reagents to the fixative solutions.

Further disclosed is a facilitated two-phase procedure for the fixation step with both cross-linking and non-cross-linking fixative agents. For cross-linking fixatives, such as formaldehyde, the invention relates to a penetration phase at cold temperature, optionally followed by a cross-linking phase at hot or ambient temperature: at cold temperature, activity of most enzymes in the ischemic cascade is suppressed; at cold temperature, cross-linking reaction is suppressed, facilitating even penetration of formaldehyde molecules throughout tissue specimens; at high temperature, cross-linking reaction is fast and reaction time can be limited in a short period facilitating standardization of cross-linking levels.

Cross-linking temperature and time can be adjusted to produce preferred level of cross-linking/modifications. For example, tissue specimens can be fixed by NBF at 4-10 degree C. for overnight with or without ultrasonic irradiation, followed by routine dehydration, clearing, and paraffin impregnation on an automatic tissue processor. Cellular and subcellular morphology of the resulting tissue sample is no different from that of tissue samples fixed at room temperature in a routine procedure. However, due to the low level of biomolecule cross-linking and modification, availability for biomolecules to extraction and detection, as well as quality and integrity of biomolecules, are significantly improved.

For non cross-linking fixatives, also called coagulant fixatives, such as alcohols, ketones, and other fixatives based on alcohols and or ketones, as well as zinc-based fixatives, at cold temperature, activity of most enzymes in the ischemic cascade is suppressed, so autolysis is prevented during penetration of fixatives into tissue specimens.

Extraneous physical forces, such as ultrasound, are applied to overcome the lack of efficient penetration of fixative molecules into tissue specimens at a cold temperature, i.e. in the range between 0 degree C. to 25 degree C., and preferably 4 degree C. to 18 degree C., in order to shorten fixation time. Since ultrasound generates moderate heat in the solution and tissue specimens in the solution it irradiates, a cooling system is needed to keep the cold temperature. The higher intensity the ultrasound irradiation is the more powerful a cooling system must be. Ultrasound irradiation can be continuous when a relative low intensity is used; or pulsate when a relative high intensity is used. When fixative molecules penetrate into tissue specimens under ultrasound irradiation, the cooling system can be turned off, and solution and tissue temperature is increased by ultrasound alone or in combination of other heating mechanisms (e.g., microwave heating, infrared heating, ohmic heating, and electronic heating). Ultrasound facilitates penetration of fixatives into tissue specimens at cold temperature; temperature is raised by ultrasound irradiation to further facilitate cross-linking or coagulation of biomolecules in tissue specimens.

In the example of formalin fixation, establishing a cold penetration protocol can significantly slow down the biomolecule changes as well as cross-linking formation during the penetration stage. Once methylene glycol molecules are evenly driven into tissue specimens, it will be possible for the succeeding cross-linking reaction to be well controlled for tissues of different type and size by a standard set of time and temperature. The new procedure for the ultrasound-facilitated tissue fixation may be as the following: 1) Immediately after removal from patients tissue specimens are placed in cold fixative in (or optionally, in a special US transparent container and transferred to) an ultrasound device with a cooling system. The tissue specimens are irradiated with ultrasound to speed up formalin penetration when the cooling system is turned on to maintain cold temperature; 2) Sound signal through and/or from tissue specimens is collected and analyzed to monitor penetration (optional); 3) When penetration is completed, the cooling system is turned off—continued ultrasound irradiation, alone or in combination with other heating system(s), brings tissue specimen temperature up to the cross-linking temperature; 4) Sound signal through and/or from tissue specimen is collected and analyzed to monitor cross-linking (optional); 5) When cross-linking is completed, fixed tissue specimens can be subjected directly to the ultrasound-facilitated processing procedure (case-by-case workflow) or lower the temperature to 4 degree C. to stop cross-linking process and wait for batched processing (routine workflow).

In the example of fixation with formalin, penetration time is dependent on tissue thickness. For tissues of thickness less than 4 mm, cold penetration time should be controlled below 60 min under ultrasound irradiation. To maintain low level of cross-linking, fixation step should be performed only at a cold temperature, with or without ultrasound irradiation. A cooling system is used to superimpose on the ultrasound irradiation when both cold temperature and ultrasound are necessary.

For tissues greater than 5 mm in thickness, fixative penetration at a cold temperature (e.g., 4-15 degree C.) may take a long time (longer than 2 hr) under ultrasound irradiation, and a temperature slope is necessary to span the range of cold penetration temperature to the cross-linking temperature (25-70 C). The time required for temperature slope for tissues thicker than 5 mm is set to be in the range of 10 min to 6 hours. Ultrasound power can be changed to adjust the heat delivered into the solution and tissue specimens in it. The cross-linking temperature is set in the range of 25 degree C. to 70 degree C., preferably in the range of 40 degree C. to 60 degree C., and further preferably in the range of 45 degree C. to 55 degree C. The cross-linking time is set to be in the range of 2 to 60 min, preferably in 5 to 15 min.

To control cross-linking/modification levels, chemical reagents or an additional cooling step are used to prevent prolonged cross-linking reactions which often leads to over cross-linking. Since properties of different tissue types differ greatly because of different structure and contents, time needed to complete fixation and processing steps differ greatly from one tissue type to another. Fixation and processing time are also closely related to thickness of the tissue specimens to be preserved as well as temperature of the preservation reagent. A predetermined time means that duration of a fixation or processing step is determined for each specific tissue type, thickness, temperature of the preservation reagent, and ultrasound parameters. According to our experiments, formalin fixation time is between 10 to 120 min for tissues of less than 3 mm in thickness at 4 to 15 degree C. under ultrasound irradiation. Fatty tissues, such as body fat and mammary gland, need longer time to be fixed.

Ultrasound and temperature control can also be used in the processing steps in tissue specimen preservation. Microcavitations caused by ultrasound irradiation leads to instant alternative high pressure and low pressure in the medium and tissue specimens therein. This effect leads to emulsification and degassing in tissues. Ultrasound also produces strong convection in the solution under irradiation, leading to good solution circulation. No stirring is necessary when ultrasound is applied. Ultrasound simultaneously delivers heat into the medium solution and therefore serves as a heating energy source.

Low pressure and optionally alternative low and high pressures applied to the fixatives and other tissue preservation reagents are helpful in improving tissue fixation and processing procedures. Tissue preservation reagents include fixation reagents (fixatives), dehydration reagents (alcohols, ketones, etc.), clearing reagents (xylene, xylene substitute), and paraffin.

Typically, tissue specimens fixed in cross-linking fixatives must undergo dehydration in alcohol to remove water. This invention discloses using ultrasound to promote the dehydration step. Temperature is an important factor in the dehydration process. The higher temperature the dehydration step is performed, the shorter time is needed, and vice versa. According to our experiments, effect of ultrasound irradiation in the dehydration step is maximized when proper temperature is reached. For fast completion of the dehydration step, the dehydration solution is maintained at 0 degree C. to 25 degree C. below the boiling temperature of the dehydration solution, and preferably 5 degree C. to 10 degree C. below boiling temperature. For example, when 100% ethanol (boiling point 78.4 degree C. under normal atmosphere pressure) is used as dehydration solution, the optimal temperature is 50 to 78 degree C. One other function of ultrasound irradiation in the dehydration step is to maintain solution temperature, alone or combined with other heating means. However, for the best morphological results moderate temperature is required, although processing time may be extended. Moderate temperature is in the range of 4 C to 50 degree C. Ultrasound irradiation can significantly shorten dehydration step at moderate temperature.

Clearing is a step in tissue processing in which the alcohol is replaced by clearing agents such as xylene or xylene substitute. Xylene and xylene substitute are inter-mixable with wax or paraffin. So the clearing step is to facilitate the next wax infiltration step. Clearing agents also function to remove fatty components from tissue. This invention discloses using ultrasound combined with temperature control to promote efficiency of the clearing step. Ultrasound is applied in the clearing step at the temperature range of 40 degree C. to 80 degree C., preferably 0 degree C. to 15 degree C. and further preferably 0 degree C. to 5 degree C. above the melting point of wax that is to be used in the next infiltration step. Some researchers use 2-propanol as a clearing agent. Since 2-propanol is poor in inter-mixing with wax, it must be evaporated from wax either by heating and/or vacuum. In this invention, we disclose that ultrasound is applied to evaporate 2-propanol from wax by its degassing and heating functions. The preferred temperature range for ultrasound irradiation to evaporate 2-propanol is 0 degree C. to 15 degree C. under its boiling temperature, which is 82.3 degree C. under normal atmosphere pressure.

In the example of preparing tissue samples with coagulant fixatives, such as alcohols, ketones, alcohol- or ketone-based fixatives, and other non-cross-linking fixatives, initial cold temperature is preferred for the fixatives. It is preferred that tissue specimens are immediately placed in the refrigerated cold fixative and kept at the cold temperature until ultrasound irradiation is started, to promote biomolecule preservation. When non-cross-linking fixative is used, no hard crust at tissue periphery caused by cross-linking of proteins will form. Fixative penetration at low temperature will function to slow down the ischemic cascade or auto degradation in tissue specimens. Ultrasound will be applied when the cooling system is turned off to raise and keep the solution temperature, alone or in combination with other heating means. According to our studies, coagulant fixatives work most effectively at the temperature range of 0 degree C. to 20 degree C. below their boiling point, with or without application of ultrasound. A temperature slope is often necessary.

Since initial penetration of fixative solutions into tissue specimens is performed at a refrigerated cold temperature where the tissue structure is in a less fluidic state, ultrasound of low frequency which causes strong cavitations can be used. Once tissue specimens are fixed, they become harder and hence can endure stronger cavitations. The US frequency range used in the disclosed invention is 100 KHz to 5 MHz, preferably 200 KHz to 2 MHz, and further preferably in the range of 0.4 MHz to 1.5 MHz. Ultrasound power is in the range of 0.1 to 100 Watts of total output, preferably between 5 to 60 Watts, further preferably between 10 to 30 Watts.

This invention also discloses a tissue sample preparation device which comprises at least one ultrasound chamber, at least one ultrasound generating and delivering device, a series of tissue fixation and processing solutions, at least one cooling device being coupled to the ultrasound chamber. Optional components include, but not limited to, the following: specimen containers holding a cold fixative, a refrigerated cabinet holding specimen containers filled with a cold fixative, at least one heating system coupled to ultrasound chamber, at least one sound signal collector, at least one sound signal analyzer, and a central processing unit (CPU).

The US reaction chambers are critical components. Besides holding solutions and samples, they are responsible for delivering the proper amount US energy to the solution in the chamber and to the tissue samples. Our study showed that a uniform distribution of US energy throughout the solution in the chamber is important for consistent fixing and processing of tissues. The efficiency of energy transmission from the transducer to the solution in the chamber, a major factor of machine efficiency and reliability, is determined by coupling efficiency. Our preferred US reaction chamber will be a container made of stainless steel or another metal alloy as a whole piece (a cup), with the transducer attached to the outside face of the bottom of the container. Another configuration of the ultrasound chamber include a metal, a plastic, a glass, or a porcelain cup holding the fixative or processing solution and a transducer housing removably submerged in the solution in the cup, the transducer housing can be configured to emit ultrasound wave from one side or from both sides. Function of the transducer housing is to seal the piezoelectric transducer from preservation reagents submerging the transducer. To emit ultrasound from one side of the transducer housing, the transducer is attached to one flat side of the housing by a glue while the other face of the transducer is unbounded. To emit ultrasound from both sides of the transducer housing, the transducer is either attached to both flat sides of the housing by a glue, or freely submerged in an inert liquid in the housing. In this case, ultrasound is transmitted through the inert liquid and both flat sides of the housing to the tissue preservation reagents. Tissue specimens can be placed on both sides of the transducer housing if ultrasound is emitted from both sides of the housing, doubling the number of tissue specimens to be preserved.

In one embodiment of formalin fixation step, the low temperature phase (0 degree C. to 15 degree C.) and the high temperature phase (35-75 degree C.) can either be performed in a same US chamber or be performed in two different US chambers each holding a fixative solution of relevant preferred temperature.

In one embodiment, vacuum can be applied to the solution and the tissue specimens in the US chamber. Vacuum can be generated by a separate vacuum pump. A moderate vacuum can also be generated by a piston-pipe structure built into the US chambers.

Conventional laboratory cooling systems include refrigerators, freezers, or even ice and dry ice. We tested the cooling effects of a 4 degree C. refrigerator, an ice-water bath, and a −20 degree C. freezer, to cool down water of 250 ml in a US chamber with US irradiation at a fixed 50 W of electronic power output. Temperature eventually could reach a stable level but could not be controlled by the operator. We disclose a temperature adjustable cooling/heating system capable of being coupled directly to the US chambers. It will comprise a refrigerated circulating bath (adjustable temperature range −20 degree to 60 degree C.) and a circulating temperature-coupling device, such as a copper pipe coil, connected to the refrigerated circulating bath by rubber tubing, or a cooling jacket. Circulating liquids can be water, ethanol, Freon. Heating can also be achieved by wrapping a heating pad around the US chamber.

It is important to insure that fixation and processing steps are adequately accomplished, especially in fast procedures. Therefore, a quality control system is necessary. When ultrasound is applied to facilitate fixation and processing, reflected and/or transmitted ultrasonic signals from tissue samples being fixed and processed can provide information to indicate the progress of each step, and an acoustic monitoring system with modest complexity can be established.

### EXAMPLES

### Example 1

We treated lysozyme and BSA with NBF for 60 min at various temperatures and immediately loaded the cross-linked products onto SDS gels for separation. For both lysozyme and BSA, cross-linking level gradually increased in the temperature range between 0 degree C. and 40 degree C. As revealed by the SDS PAGE, there is a significant increase in size of oligomers (lysozyme) or in gel mobility (BSA) at 50 degree C., which peaks at around 60 degree C. The inventors noticed that large amount of aggregates began to form when incubating lysozyme with NBF at a temperature greater than 60 degree C. These aggregates were highly intermolecularly cross-linked lysozyme molecules, and could not be separated on SDS PAGE. No aggregates were observed with BSA in NBF at high temperatures, possibly due to the fact that cross-linking in BSA was mostly intramolecular.

In the case of lysozyme, there was a gradual acceleration of cross-linking speed at temperature of 0-20 degree C. and leveled at the range of 20-40 degree C. which then followed by abrupt speed increase at temperatures 50 degree C. and up. While in the case of BSA, cross-linking gradually accelerated at temperatures from 0 degree C. to 30 degree C. and abruptly accelerated at temperatures 40 degree C. Experiments with these two typical proteins showed that speed of cross-linking formation in formalin fixation accelerated with increase in temperature, and speed acceleration may be classified into two modes according to incubation temperature: 1) slow acceleration which happens at 0 degree C.-40 degree C., and, 2) fast acceleration which happens at 40-100 degree C. With both proteins, we speculate that temperature between 50 degree C. and 60 degree C. is an important range at which cross-linking happens at high but controllable speed. At temperatures higher than 60 degree C., insoluble aggregations caused by intermolecular cross-linking began to form in large amount, as revealed by the experiment with lysozyme.

### Example 2

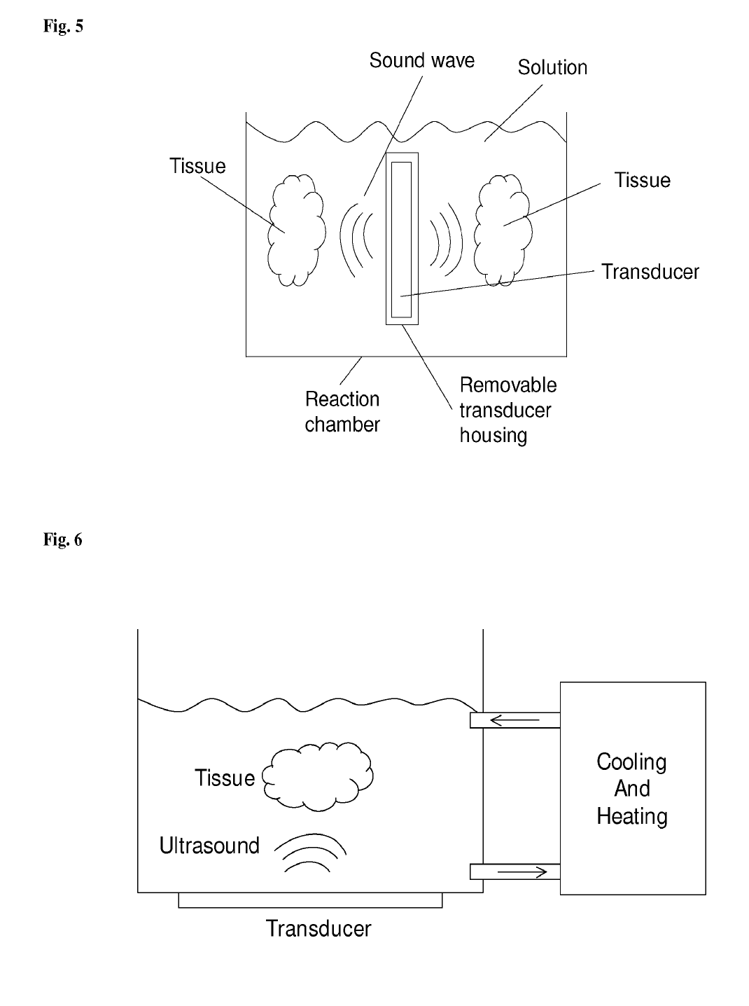

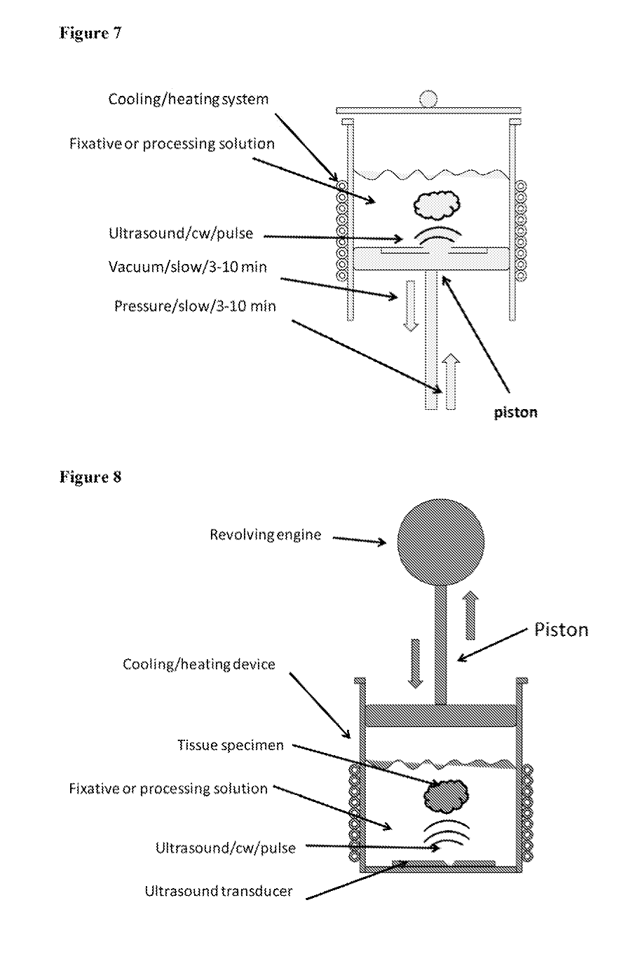

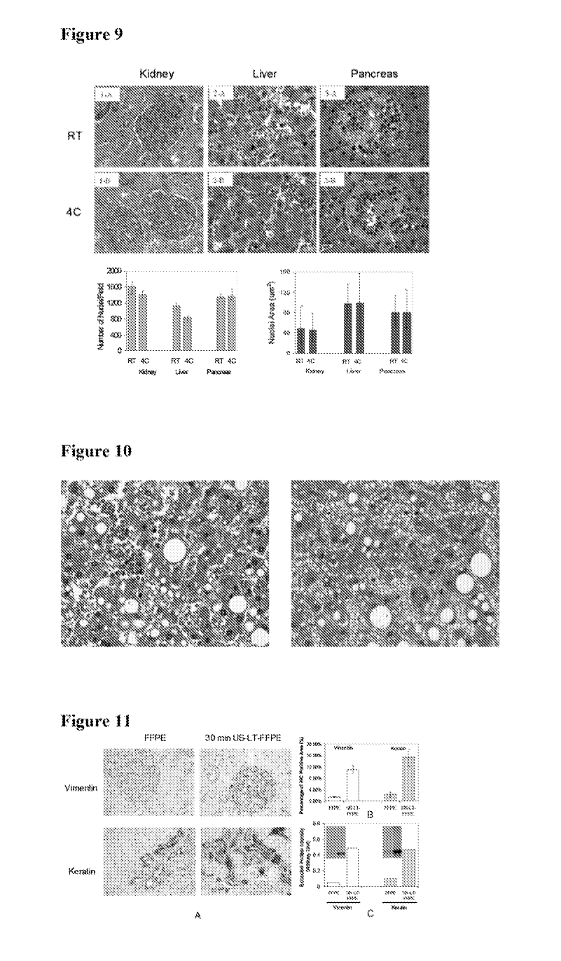

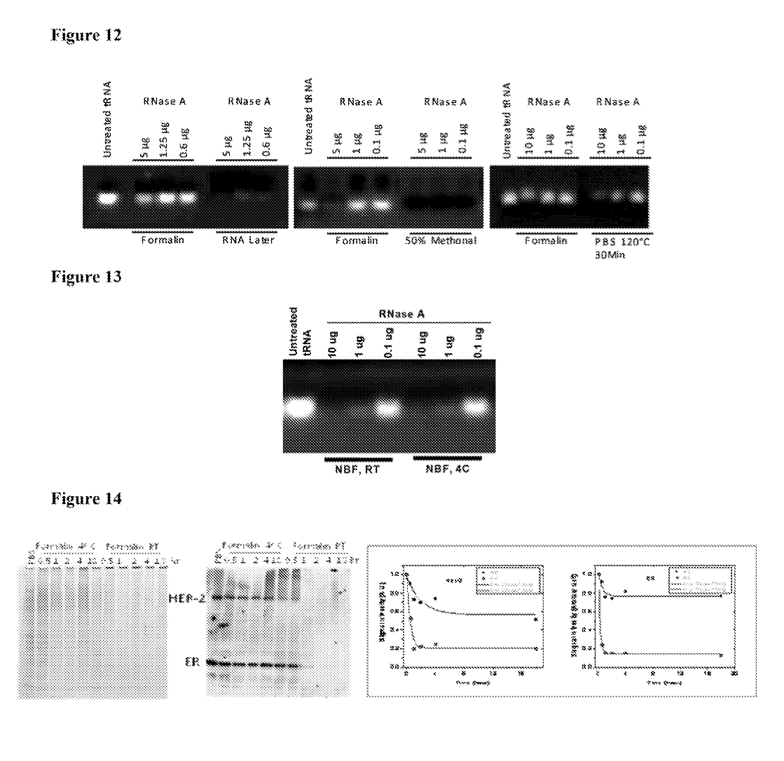

Significant cross-linking occurs after 10-minute incubation at 50 degree C. In another experiment, we tested cross-linking formation for various proteins when incubated in formalin at 4 degree C., RT, and 50 degree C. for 10 min, in comparison with incubation with formalin at RT overnight. As shown in FIG. 5, cross-linking at 4 degree C. was the slowest for all the proteins tested. And there is a big increase in levels of cross-linking after incubation at 50 degree C. for 10 min, almost comparable to the cross-linking levels after overnight incubation at room temperature. For all proteins except BSA, protein precipitations formed when incubated with formalin at 50 degree C. for 10 min and at RT overnight. This experiment indicated that for many proteins incubation with formalin at 50° C. for 10 minutes lead to significant cross-linking formation. Similar results were also obtained for 5-minute incubations (data not shown).

### Example 3

Tissue specimens of 1-4 mm thick were held in tissue cassettes and submerged in a cross-linking fixative (e.g. formalin, 4-25 degree C.) until ultrasound irradiation. Ultrasound is applied to bring the fixative temperature up to 50-80 degree C. alone or in combination with other heating means. The temperature was maintained for 5 to 20 min.

The tissue specimens were then submerged in a dehydration agent, e.g., 100% ethanol. Temperature of the dehydration agent was brought up to 50-75 degree C. by ultrasound irradiation alone or in combination with another heating means. The temperature was maintained by ultrasound irradiation alone or in combination of another heating means for 5-30 min. This step was repeated at least once.

The tissue specimens were then submerged in a clearing agent, e.g., xylene or xylene substitute. Temperature of the clearing agent was brought up to 50-75 degree C. by ultrasound irradiation alone or in combination with another heating means. The temperature was maintained by ultrasound irradiation alone or in combination of another heating means for 5-30 min. This step was repeated at least once.

The tissue specimens were then submerged in melted wax or paraffin at a temperature range of 55-70 degree C. Ultrasound irradiation was applied to melted wax or paraffin for 5-30 min. Temperature of the wax or paraffin was maintained at 60-75 degree C. by ultrasound irradiation alone or in combination with another heating means.

### Example 4

Tissue specimens of 1-4 mm in thickness were held in tissue cassettes and submerged in a non-cross-linking fixative (e.g., 100% ethanol, acetone, or a mixture of 40% isopropyl alcohol, 40% acetone, 20% polyethylene glycol (average molecular weight 300) and 1% dimethyl sulfoxide (DMSO)) at the temperature range of 4-25 degree C. Ultrasound was applied to bring temperature to the range of 50-70 degree C. alone or in combination with another heating means. The temperature was maintained for 5 to 20 min. Dehydration, clearing, and wax infiltration steps were performed as described in Example 6.

### Example 5

Tissue specimens of 3 mm or thinner were held in tissue cassettes and submerged in NBF at temperature of 50-70 degree C. Ultrasound is applied for 5-15 minutes and temperature of NBF is maintained by ultrasound irradiation alone or in combination with another heating means. Dehydration, clearing, and wax infiltration steps were performed as described in Example 6.

### Example 6

Fresh tissue specimens of 5 mm or thicker were submerged in NBF at a temperature range of 4-10 degree C. Ultrasound was applied to NBF. To maintain temperature within 4-10 degree C. a cooling system was applied to cool down NBF. Ultrasound irradiation lasted for at least 30 minutes.

The cooling system was turned off. NBF temperature was raised by continued ultrasound irradiation until temperature reached the range of 55-80 degree. Temperature was maintained by ultrasound irradiation alone or in combination with a heating pad for 10-30 minutes.

### Example 7

We fixed fresh bovine kidney, liver, and pancreas tissues (3 mm×5 mm×5 mm) in formalin overnight at 4 degree C. or room temperature and processed the fixed tissues on an automatic tissue processor. Tissues fixed overnight at 4 C still appeared fresh while tissues fixed overnight at RT were grayish. 3 micron sections from the FFPE paraffin blocks were stained by H&E and viewed under a microscope. Cell morphology in tissue samples fixed at 4 degree C. generally had brighter and sharper edge as well as better sub-cellular details. Low temperature fixation greatly reduced vacuolization commonly existing in tissues fixed by the routine fixation procedure. Red blood cell lysis was also reduced in tissues fixed in cold formalin. The most striking difference between the two fixation methods is demonstrated in pancreas samples. Pancreas tissues fixed in RT formalin overnight showed overt destruction in cellular structures in the islets. Number of nuclei per field and nuclear size are calculated to analyze whether there is any cellular or intracellular shrinkage due to low temperature. The quantitative counting results showed no detectable change due to the low temperature; contrary to a previous study with liver tissue that formalin fixation at 4 degree C. overnight resulted in tissue shrinkage [Fox 1987]. Experiment with the human ovary cancer tissue specimen also showed that fixation at 4 degree C. produced better morphological features than those by the conventional room temperature method.

### Example 8

We studied US effect in promoting diffusion of formalin molecules into tissue specimens at a refrigerated temperature using human ovary and liver tissues. Fixation at 4 degree C. for 18 hr generated better morphological features than fixation at 8 degree C. for 5 min under US, which is better than the conventional RT fixation for 18 hr.

Human liver tissue (3 mm thickness) was fixed in 4 degree C. formalin with US irradiation for 30 minutes. As a control, the same liver tissue of similar size was fixed in 4 degree C. formalin for 30 minutes without US. The liver tissue fixed with US irradiation showed good morphological features. While the liver tissue fixed without US irradiation demonstrated severe loss of red blood cell integrity and subcellular structure details. Since the succeeding processing steps were done through regular alcohol dehydration, xylene clearing and paraffin embedding, the tissue fixed in 4 degree C. formalin for 30 min without US produced alcohol fixation morphology while the tissue fixed in 4 degree C. formalin for 30 min with US produced excellent formalin fixation morphology.

### Example 9

Cow kidney tissue (3 mm thickness) was fixed in 4 degree C. formalin for 30 min with US irradiation (US-LT-FFPE) or at RT overnight (FFPE), and then processed on automatic tissue processor. IHC was carried out for vimentin and cytokeratin without the antigen retrieval (AR) process. In both IHC assays, the sample fixed with US irradiation produced better results than the routine FFPE sample. The percentage of IHC stain positive area in US-LT-FFPE indicates that more antigen epitopes (antigenic determinants) are preserved than routine FFPE methods. Protein extracted from tissue specimens also showed that US facilitated fixation at 4 degree C. resulted in stronger signals for both vimentin and cytokeratin than routine FFPE for Western blot. This data indicates that antigens in the US-LT-FFPE tissue sample are modified to a lesser degree and more available for IHC detection and extraction than the routine counterpart because of reduced cross-linking levels.

### Example 10

To study if US can facilitate formalin fixation of large tissues, we fixed a human autopsy whole prostate specimen in formalin with US irradiation at 10 degree C. for 16 h and at 25 degree C. for 2 h. As a control, a surgically removed patient's whole prostate specimen was fixed in formalin at RT. After the 18-h fixation, the prostate specimen fixed by the US method still looked fresh with blood, fat, and capsule tissues maintaining their original appearances. The prostate specimen that was fixed overnight by the conventional method looked gray. We roughly compared hardness of both prostates by touching them with our fingers. The prostate fixed by the US method appeared to be harder than the one fixed by the conventional method.

Both prostate specimens were subjected to an MRI examination after overnight fixation. The prostate fixed by the US method generated darker and more even images on every plane selected in both the peripheral and center regions while the conventionally fixed prostate generated uneven images with scattered bright spots in the center region. The dark areas in an MRI image are correlated with the density of the tissue specimen [Hennig 1986]. Cross-linking caused by formaldehyde creates a density change in tissue specimens that can be revealed by MRI.

### Example 11

We have designed a series of direct forward biochemistry experiments to compare various chemical reagents such as formalin, RNA Later, methanol/alcohol, as well as heating conditions (boiling or autoclaving) in inhibiting the RNase bioactivity. The study results indicate that formalin is the best RNase inhibitor. We also compare the inhibition ability of formalin at room temperature and 4 degree C. No difference was found under this comparison study. This result suggests that instant formalin inhibition of the RNase activity at 4 degree C. is not dependent on slow cross-link reaction. This is the primary data on free solution reaction, however, the preservation of RNA by cold formalin at tissue level should be further studied. According to the cell protein and RNase experimental results, we suggest that the low temperature formalin fixation can achieve the similar enzyme inhibition ability and it can preserve the biomolecules with low modification (low level cross-linking).

### Example 12

To make a rough comparison on proteins extractable from samples fixed at 4 degree C. and RT, we did a time course study measuring 2 specific proteins with Western blot using cultured cells. Diffusion time of formalin molecules into cultured cells can be neglected, in this case incubation time can better represent the cross-linking time. Aliquots of freshly grown breast cancer T47D cells were treated with neutral buffered formalin (NBF) at 4 degree C. or RT for different lengths of time. Proteins in cell lysates were separated by SDS PAGE and then transferred to a nitrocellulose membrane which was probed with pooled monoclonal antibodies against HER-2 and ER, a membrane protein and a nuclear protein respectively. Coomassie blue stained SDS gel showed that cells fixed at 4 degree C. generated more proteins lysates throughout fixation time 0.5 to 18 hr) than cells fixed at RT. Western blot showed that at 4 degree C. fixation conditions similar signal strengths for HER-2 and ER proteins were produced for fixation times between 0.5 to 4 hr, while at RT only the 0.5 hr fixation time produced readable signals for both proteins. Strikingly, we found that, in terms of protein extractability, RT fixation for 0.5 hr is roughly equivalent to 4 degree C. fixation for 18 hr.

### REFERENCES

1. Auer H, Lyianarachchi S, Newsom D, Klisovic MI, Marcucci G, Kornacker K. Chipping away at the chip bias: RNA degradation in microarray analysis. Nat Genet. 2003. 35(4):292-293.

2. Bancroft JD, Stevens A, Turner DR. Theory and practice of histological techniques. New York, Churchill Livingstone, 1996.

3. Boon ME, Gerrits PO, Moorlag HE, Nieuwenhuis P, Kok LP. Formaldehyde fixation and microwave irradiation. Histochem. J. 1988, 20: 313-322.

4. Boon ME, Kok LP. Theory and practice of combining coagulant fixation and microwave histoprocessing. Biotech Histochem. 2008. 83(6):261-77.

5. Chu W, Furusato B, Wong K, Mostofi FK, Zhu Z, Abbondanzo SL, and Liang Q. Wei MQ, Sesterhenn Iowa, Ultrasound-Accelerated Formalin Fixation of Tissue Improves Morphology, Antigen and mRNA Preservation. Mod. Pathol. 2005, 18:850-863.

6. Chu WS, Liang Q, Tang Y, King R, Wong K, Gong M, Wei M, Liu J, Feng SH, Lo SC, Andriko JA, Orr M. Ultrasound-accelerated tissue fixation/processing achieves superior morphology and macromolecule integrity with storage stability. J. Histochem. Cytochem. 2006, 54:503-513.

7. Chu, WS, Liang Q, Liu J, Wei MQ, Winters M, Liotta LA, Sandberg G, Gong M (2005) “A Nondestructive Molecule Extraction Method Allowing Morphological and Molecular Analyses Using a Single Tissue Section” Lab. Invest. 85(11):1416-28.

8. Compton C. Getting to personalized cancer medicine—taking out the garbage. Cancer. 2007, 110:1641-1643.

9. Cronin M, Sangli C, Liu ML, Pho M, Dutta D, Nguyen A, Jeong J, Wu J, Langone KC, Watson D. 2007. Analytical validation of the Oncotype DX genomic diagnostic test for recurrence prognosis and therapeutic response prediction in node-negative, estrogen receptor-positive breast cancer. Clin Chem. 53:1084-1091.

10. Dapson RW. Fixation for the 1990's: a review of needs and accomplishments. Biotech Histochem 1993. 68:75.

11. Drakhli E. I. Methods of using ultrasonics in a quick histological treatment of tissues. Arkh. Patol. 1967, 29:81-82.

12. Emmert-Buck MR, Strausberg RL, Krizman DB, et al. Molecular profiling of clinical tissue specimens: feasibility and applications. Am J Pathol. 2000, 156:1109-1115.

13. Espina V, Edmiston KH, Heiby M, Pierobon M, Sciro M, Merritt B, Banks S, Deng J, VanMeter AJ, Geho DH, Pastore L, Sennesh J, Petricoin EF 3rd, Liotta LA. (2008) A portrait of tissue phosphoprotein stability in the clinical tissue procurement process. Mol Cell Proteomics. 7(10):1998-2018.

14. Espina V, Mueller C, Edmiston K, Sciro M, Petricoin EF, Liotta LA. Tissue is alive: New technologies are needed to address the problems of protein biomarker pre-analytical variability. Proteomics Clin Appl. 2009. 3(8):874-882.

15. Fox, C. H., Johnson, F. B., Whiting, J., and Roller, P. P.: Formaldehyde fixation. J HistocheCytochem 1985, 33:845-853.

16. Fox CH, Beran M: Formaldehyde: The Fixative. The J Histotechnol 1987, 10:199-201.

17. Helander KG. Kinetic studies of formaldehyde binding in tissue. Biotech Histochem. 1994, 69:177-179.

18. Helander KG. Formaldehyde binding in brain and kidney: a kinetic study of fixation. The J. of Histotechnology. 1999, 22: 317-318.

19. Hennig J, Nauerth A, Friedburg H. RARE imaging: a fast imaging method for clinical MR. Magn Reson Med 1986; 3:823-833.

20. Hewitt SM, Lewis FA, Cao Y, Conrad RC, Cronin M, Danenberg KD, Goralski TJ, Langmore JP, Raja RG, Williams PM, Palma JF, Warrington JA. Tissue handling and specimen preparation in surgical pathology: issues concerning the recovery of nucleic acids from formalin-fixed, paraffin-embedded tissue. Arch Pathol Lab Med. 2008, 132:1929-35.

21. Levene A. On the histological diagnosis and prognosis of malignant melanoma. J. Clin. Pathol. 1980, 33(2): 101-124.

22. Liotta LA, Petricoin E: Molecular profiling of human cancer. Nature Reviews Genetics. 2000, 1:48-56.

23. Medawar PB. The rate of penetration of fixatives. J Royal Micr Soc. 1941, 61:46-57.

24. Obertyshev V. G. Ultrasonic express paraffin handling of histological specimens. Sud. Med. Ekspert. 1987, 30:56-58.

25. Paik S, Kim CY, Song YK, Kim WS. 2005. Technology insight: Application of molecular techniques to formalin-fixed paraffin-embedded tissues from breast cancer. Nat Clin Pract Oncol. 2:246-254.

26. Pusztai L. Current status of prognostic profiling in breast cancer. Oncologist. 2008, 13:350-60.

27. Rait VK, O'Leary TJ, Mason JT. Modeling formalin fixation and antigen retrieval with bovine pancreatic ribonuclease A: 1-structural and functional alterations. Lab Invest. 2004, 84:292-299.

28. Rait VK, Xu L, O'Leary TJ, Mason JT. Modeling formalin fixation and antigen retrieval with bovine pancreatic RNase A II. Interrelationship of cross-linking, immunoreactivity, and heat treatment. Lab Invest. 2004, 84:300-306.

29. Ruijter ET, Miller GJ, Aalders TW, van de Kaa CA, Schalken JA, Debruyne FM, Boon ME. Rapid microwave-stimulated fixation of entire prostatectomy specimens. Biomed-II MPC Study Group. J Pathol. 1997, 183(3):369-75.

30. Schroeder A, Mueller O, Stocker S, Salowsky R, Leiber M, Gassmann M, Lightfoot S, Menzel W, Granzow M, Ragg T. The RIN: an RNA integrity number for assigning integrity values to RNA measurements. BMC Mol Biol. 2006. 31;7:3.

31. Shmurun RI. Methods for the rapid preparation of paraffin blocks. Arkh Pathol. 1992, 54: 46-47.

32. Stephanis CG, Hatiris JG, and Mourmouras DE. Acceleration of formaldehyde reactions with proteins due to ultrasound. Ultrasonics Sonochemistry. 1998. 5:33-35.

33. Wenk P. Fixatives: coagulant vs non-coagulant or is it additive vs non-additive? Histologic 2006. 39:36

34. Wester K, Asplund A, Backvall H et al. Zinc-based fixative improves preservation of genomic DNA and proteins in histoprocessing of human tissues. Lab Invest 2003. 83:889

1. A method of preserving tissue specimens comprising:
a) submerging said tissue specimens into a fixative of a temperature between 0 to 25 degree C.,
b) subjecting said tissue specimens and said fixative to a cooling mechanism, and,
c) subjecting said tissue specimens and said fixative to a convection mechanism.

2. The method of claim 1, wherein said fixative is a formaldehyde water solution comprising 1% to 10% of formaldehyde by weight, and wherein said temperature is between 4 to 20 degree C., preferably between 4 to 18 degree C.

3. The method of claim 1, wherein said fixative is a solution comprising at least 50% by volume of at least one chemical from the following group: ethanol, methanol, PEG, and acetone; and
wherein said temperature is between 4 to 25 degree C., preferably between 4 to 18 degree C.

4. The method of claim 1, wherein said convection mechanism is ultrasound irradiation.

5. The method of claim 4, wherein said ultrasound is of a frequency between 0.1 MHz to 5 MHz, preferably between 0.2 MHz to 2 MHz, and further preferably between 0.4 MHz to 1.5 MHz.

6. The method of claim 5, wherein said ultrasound is of a power between 1 to 100 Watts, preferably between 5 to 50 Watts, and further preferably between 10 to 30 Watts.

7. A method of claim 6 further comprising at least one dehydration step of submerging fixed tissue specimens in a dehydration solution of a temperature between 0-25 degree C.

8. A method of claim 6, further comprising: d) turning off said cooling mechanism after a predetermined length of time; e) continuing ultrasound irradiation to increase temperature of said tissue specimens in said fixative to a predetermined value; f) switching ultrasound irradiation on and off to maintain said predetermined temperature value for a predetermined length of time.

9. A method of claim 6 further comprising:
at least one dehydration step; comprising: i) submerging fixed tissue specimen in a dehydration solution of a temperature between 0-40 degree C., ii) raising the temperature of said dehydration solution to a predetermined value by ultrasound irradiation alone or in combination with other heating means, and, iii) keeping the temperature value for a predetermined length of time;
at least one clearing step, comprising: i) submerging dehydrated tissue specimen in a clearing solution, ii) raising the temperature of said clearing solution to a predetermined value by ultrasound irradiation alone or in combination with other heating means, and, iii) keeping the temperature for a predetermined length of time:
an impregnating step, comprising: i) submerging the dehydrated and cleared tissue specimen in melted wax, ii) irradiating said dehydrated and cleared tissue specimen in melted wax with ultrasound for a predetermined length of time, and, iv) applying a vacuum to said dehydrated and cleared tissue specimen in melted wax.

10. The method of claim 1, wherein said convection mechanism is by stirring, agitation, or pumping-in and pumping-out.

11. The method of claim 1, further comprising:
d) subjecting said tissue specimens and said fixative to low pressure, wherein said low pressure is in the range of 10 to 20 inch Hg.

12. The method of claim 1, further comprising:
d) subjecting said tissue specimens and said fixative to high pressure, wherein said high pressure is a pressure that is above atmospheric air pressure.

13. The method of claim 1, further comprising:
d) subjecting said tissue specimens and said fixative to alternative high pressure and low pressure.

14. A device for preserving tissue specimens in an ultrasound field, comprising:
1) At least one vessel for holding tissue specimens and preservation reagent(s),
2) An ultrasound generating system to produce ultrasound field in reagent(s), and
3) A temperature control system for maintaining a preset temperature in said vessel.

15. A device of claim 14, wherein said ultrasound generating system produces ultrasound signal of:
1) a frequency in the range of 0.1 MHz and 5 MHz, preferably in the range of 0.2 MHz and 2 MHz, further preferably in the range of 0.4 MHz and 1.5 MHz; and, 2) a power in the range of 0.1 to 100 Watts, preferably between 5 to 50 Watts, further preferably between 10 to 30 Watts.

16. A device of claim 15, wherein said ultrasound generating system comprises at least one piece of piezoelectric transducer, said piezoelectric transducer can be in the shape of a circular disc, a rectangle bar, a cylinder, or an array of multiple transducers.

17. A device of claim 16, wherein said piezoelectric transducer(s) is attached to the surface of the wall or the bottom of said vessel by a glue. 26

18. A device of claim 16, wherein said piezoelectric transducer (s) is sealed in a transducer housing.

19. A device of claim 18, wherein said transducer housing is submerged in said tissue preservation reagent(s).

20. A device of claim 18, wherein ultrasound is produced from one side of said transducer housing to irradiate on tissue specimens that are placed on the same side of said transducer housing emitting ultrasound.

21. A device of claim 18, wherein ultrasound is produced from both sides of said transducer housing to irradiate on tissue specimens that are placed on both sides of said transducer housing.

22. A device of claim 15, wherein said ultrasound generating system can deliver continuous or pulsed ultrasound field with fixed or sweep frequency(s).

23. A device of claim 14, wherein said vessel contains a side wall, a detachable cover, and a movable bottom fitted water tight to the side wall; said movable bottom is connected with a revolving engine; said revolving engine drives back and forth movements of said moveable bottom.

24. A device of claim 14, wherein said vessel contains a side wall, a bottom, and a movable cover;
said movable cover is fitted water tight to the side wall and is connected with a revolving engine; said revolving engine drives back and forth movements of said movable cover.

25. A device of claim 14, wherein said temperature control system contains a cooling means and a heating means.

26. A device of claim 24, wherein said cooling means is circulating a cold liquid through a space surrounding said vessel; said space is formed between the outside surface of said vessel and a shell surrounding said outside surface of said vessel; and said cold liquid is of a temperature between −20 degree to 25 degree C.

27. A device of claim 24, wherein said cooling means is circulating a cold liquid through a pipe passing through said tissue preservation reagent.

28. A device of claim 24, wherein said cooling means is a semiconductor device.

29. A device of claim 24, wherein said heating means is ultrasound irradiation alone or in combination of ohmic heating or microwave heating.
